(19) United States
(12) Patent Application Publication
(10) Pub. No.: US 2012/0315666 A1
(43) Pub. Date: Dec. 13, 2012

(54) DETECTION APPARATUS AND METHOD FOR DETECTING AIRBORNE BIOLOGICAL PARTICLES

(76) Inventors: Kazushi Fujioka, Osaka-shi (JP); Kazuo Ban, Osaka-shi (JP); Norie Matsui, Osaka-shi (JP); Hiroki Okuno, Osaka-shi (JP)

(21) Appl. No.: 13/581,224

(22) PCT Filed: Jul. 7, 2010
(86) PCT No.: PCT/JP2010/004434

§ 371 Date: Aug. 24, 2012

(30) Foreign Application Priority Data
Feb. 26, 2010 (JP) 2010-042869
Feb. 26, 2010 (JP) 2010-042870

(51) Int. Cl. G01N 21/64; C12Q 1/06; C12M 1/34

(52) U.S. Cl. 435/39; 250/458.1; 250/461.2; 250/459.1; 435/288.7

(57) ABSTRACT

In a detection apparatus, an inlet and an outlet are opened and an air introducing mechanism is driven to introduce air to a case, and airborne particles are electrically attracted and held on a collecting jig 12. After introduction, the inlet and outlet are closed, and amount of fluorescence received by a light receiving element resulting from irradiation with light emitted from a light emitting element is measured by a measuring unit. Thereafter, the collecting jig is heated by a heater and the amount of fluorescence after heating is measured by the measuring unit. Based on the amount of change in the amount of fluorescence before and after heating, the amount of microorganisms collected by the collecting jig is calculated at the measuring unit.

### TECHNICAL FIELD

The present invention relates to detection apparatus and method and, more specifically, to detection apparatus and method for detecting airborne biological particles.

### BACKGROUND ART

Conventionally, for detecting airborne microorganisms, first, airborne microorganisms are collected by sedimentation, impaction, slit method, using perforated plate, centrifugal impaction, impinger or filteration and, thereafter, the microorganisms are cultivated and the number of appeared colonies is counted. By such a method, however, two or three days are necessary for cultivation and, therefore, detection on real-time basis is difficult. Therefore, recently, apparatuses for measuring numbers by irradiating airborne microorganisms with ultraviolet ray and detecting light emitted from microorganisms have been proposed, for example, in Japanese Patent Laying-Open No. 2003-38163 (Patent Document 1) and Japanese Patent National Publication No. 2008-508527 (Patent Document 2).

In conventional apparatuses such as proposed in Patent Documents 1 and 2, as means for determining whether the suspended particles are of biological origin, a method has been used in which whether or not the particle emits fluorescence when irradiated with ultraviolet ray is determined.

### CITATION LIST Patent Literature

PTL 1: Japanese Patent Laying-Open No. 2003-38163

PTL 2: Japanese Patent National Publication No. 2008-508527

### SUMMARY OF INVENTION

### Technical Problem

Actually, however, dust suspended in the air includes much lint of chemical fibers that emits fluorescence when irradiated with ultraviolet ray. Therefore, when the conventional apparatus such as proposed in Patent Documents 1 and 2 is used, not only airborne particles of biological origin but also fluorescence-emitting dust are detected. Specifically, the conventional apparatuses such as proposed in Patent Documents 1 and 2 have a problem that accurate evaluation of only the biological particles suspended in the air is impossible.

The present invention is made in view of the problem and its object is to provide a detection apparatus and method that utilize fluorescence and capable of detecting, on real-time basis, only the biological particles separate from fluorescence-emitting dust.

### Solution to Problem

In order to attain the above-described object, according to an aspect, the present invention provides a detection apparatus for detecting airborne particles of biological origin, including: a light emitting element; a light receiving element for receiving fluorescence; and a calculating unit for calculating, based on an amount of fluorescence received by the light receiving element when air introduced to the detection apparatus is irradiated with light emitted from the light emitting element, an amount of particles of biological origin in the air of a fixed amount.

Preferably, the calculating unit calculates, based on a change in the amount of received light before and after heating the particles, an amount of particles of biological origin in the introduced air.

More preferably, the detection apparatus further includes a heater for heating the introduced air.

More preferably, the detection apparatus further includes a control unit for controlling an amount of heating by the heater.

More preferably, the detection apparatus further includes an input unit for inputting an instruction to the control unit.

Preferably, the calculating unit calculates, based on a change in the amount of received light, and on a relation between the amount of change in fluorescence and the amount of particles of biological origin stored in advance, an amount of particles of biological origin in the introduced air.

Preferably, the detection apparatus further includes: a collecting member; and a collecting mechanism for collecting particles in the introduced air by the collecting member. The calculating unit calculates, based on the amount of received fluorescence from the collecting member irradiated with light emitted from the light emitting element, an amount of particles of biological origin collected by the collecting member.

More preferably, the light emitting element is arranged such that light is emitted in a direction toward the collecting member.

More preferably, the detection apparatus further includes a heater for heating the collecting member, and the calculating unit calculates, based on a change in the amount of received light before and after heating of the collecting member, an amount of particles of biological origin collected by the collecting member.

Preferably, the detection apparatus further includes a collection chamber housing the collecting mechanism, a detection chamber separated from the collection chamber and housing the light emitting element and the light receiving element, and a moving mechanism for moving the collecting member positioned in the collection chamber to the detection chamber, and for moving the collecting member positioned in the detection chamber to the collection chamber.

Preferably, the detection apparatus further includes a cleaning unit for cleaning the collecting member.

Preferably, the detection apparatus further includes a display unit for displaying a result of calculation by the calculating unit as a result of measurement.

Preferably, the light emitting element emits light in a wavelength range that can excite substance in a living organism. More preferably, the light emitting element emits light in a wavelength range of 300 nm to 450 nm.

According to another aspect, the present invention provides a method of detecting particles of biological origin collected by a collecting member, including the steps of: measuring amount of fluorescence of the collecting member before heating, irradiated with light emitted from a light emitting element; measuring amount of fluorescence of the collecting member after heating, irradiated with light emitted from the light emitting element; and calculating an amount of particles of biological origin collected by the collecting member, based on an amount of change in the amount of fluorescence measured from the collecting member before heating and the amount of fluorescence measured from the collecting member after heating.

### Advantageous Effects of Invention

By the preset invention, it becomes possible to detect biological particles separate from fluorescence-emitting dust on real-time basis with high accuracy.

### BRIEF DESCRIPTION OF DRAWINGS

FIG. 1 shows an appearance of an exemplary air purifier as the detection apparatus in accordance with an embodiment.

FIG. 2A shows a basic configuration of a detection apparatus in accordance with a first embodiment.

FIG. 2B shows a specific example of a structure around a collecting jig and a heater, in the detection apparatus in accordance with an embodiment.

FIG. 3A is an illustration of a detecting mechanism in the detection apparatus in accordance with the first embodiment.

FIG. 3B is an illustration of a detecting mechanism in the detection apparatus in accordance with the first embodiment.

FIG. 4A is an illustration of a mechanism provided at an inlet as another specific example of a light intercepting mechanism in the detecting mechanism.

FIG. 4B is an illustration of a mechanism provided at an outlet as another specific example of the light intercepting mechanism in the detecting mechanism.

FIG. 4C shows a specific example of one of light shielding plates included in each of the mechanisms provided at the inlet and outlet as another specific example of the light intercepting mechanism in the detecting mechanism.

FIG. 4D shows another specific example of one of light shielding plates included in each of the mechanisms provided at the inlet and outlet as another specific example of the light intercepting mechanism in the detecting mechanism.

FIG. 5 shows time change of fluorescent spectrum of Escherichia coli before and after heat treatment.

FIG. 6A is a fluorescent micrograph of Escherichia coli before heat treatment.

FIG. 6B is a fluorescent micrograph of Escherichia coli after heat treatment.

FIG. 7 shows time change of fluorescent spectrum of Bacillius subtilis before and after heat treatment.

FIG. 8A is a fluorescent micrograph of Bacillius subtilis before heat treatment.

FIG. 8B is a fluorescent micrograph of Bacillius subtilis after heat treatment.

FIG. 9 shows time change of fluorescent spectrum of Penicillium before and after heat treatment.

FIG. 10A is a fluorescent micrograph of Penicillium before heat treatment.

FIG. 10B is a fluorescent micrograph of Penicillium after heat treatment.

FIG. 11A is a fluorescent micrograph of cedar pollen before heat treatment.

FIG. 11B is a fluorescent micrograph of cedar pollen after heat treatment.

FIG. 12A shows time change of fluorescent spectrum of fluorescence-emitting dust before heat treatment.

FIG. 12B shows time change of fluorescent spectrum of fluorescence-emitting dust after heat treatment.

FIG. 13A is a fluorescent micrograph of fluorescence-emitting dust before heat treatment.

FIG. 13B is a fluorescent micrograph of fluorescence-emitting dust after heat treatment.

FIG. 14 shows results of comparison of fluorescent spectra of fluorescence-emitting dust before and after heat treatment.

FIG. 15 is a block diagram showing an exemplary functional configuration of the detection apparatus in accordance with the first embodiment.

FIG. 16 is a time-chart showing a flow of operations in the detection apparatus in accordance with the first embodiment.

FIG. 17 is a graph showing specific relation between fluorescence decay and microorganism concentration.

FIG. 18A shows an exemplary display of detection results.

FIG. 18B shows a method of displaying detection results.

FIG. 19 shows a basic structure of the detection apparatus in accordance with a second embodiment.

FIG. 20 is an illustration related to an operation of a collecting unit of the detection apparatus in accordance with the second embodiment.

FIG. 21 is a time-chart showing a flow of operations in the detection apparatus in accordance with the second embodiment.

FIG. 22 schematically shows a configuration of an instrument used by the present inventors for measurement.

FIG. 23 shows a result of measurement in an example 1.

FIG. 24 shows a result of measurement in an example 2.

FIG. 25 shows a relationship between temperature of a heat treatment of Penicillium and a ratio of intensity of fluorescence provided from Penicillium before and after the heat treatment.

### DESCRIPTION OF EMBODIMENTS

In the following, embodiments of the present invention will be described with reference to the figures. In the following, the same parts and components are denoted by the same reference characters. Their names and functions are also the same.

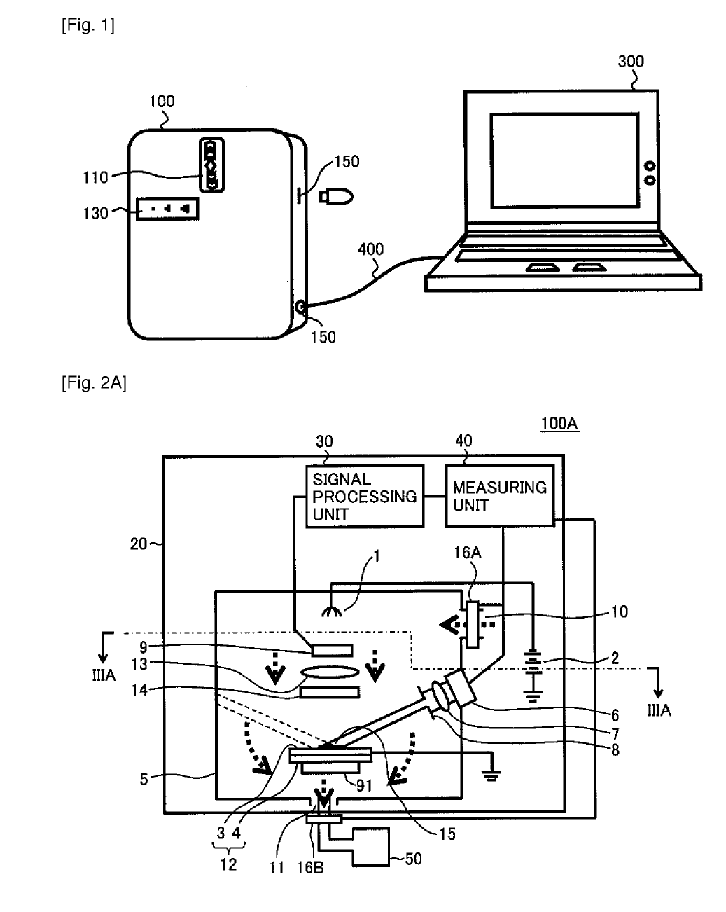

In the embodiments, it is assumed that the air purifier shown in FIG. 1 functions as a detection apparatus. Referring to FIG. 1, the air purifier as detection apparatus 100 includes a switch for receiving an operation instruction, and a display panel 130 for displaying detection results and the like. Further, a suction opening for introducing air and an exhaust opening for discharging air, not shown, are provided. Detection apparatus 100 further includes a communication unit 150 to which a recording medium is attached. Communication unit 150 may provide connection to a personal computer (PC) 300 as an external apparatus using a cable 400. Alternatively, communication unit 150 may provide connection to a communication line for communication with other apparatuses through the Internet. Communication unit 150 may communicate with other apparatuses through infrared communication or through the Internet.

### First Embodiment

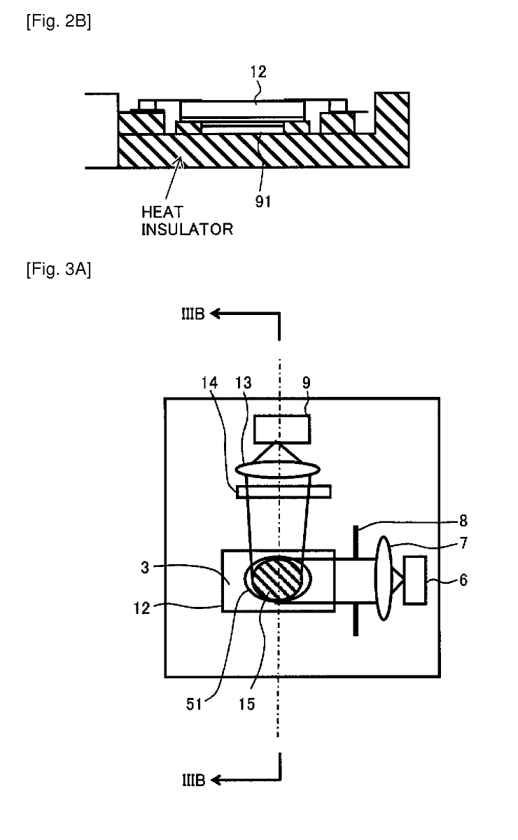

Referring to FIG. 2A, a detection apparatus 100A in accordance with the first embodiment, which is of a detection apparatus 100 according to an embodiment that is a detecting apparatus portion of the air purifier, has a case 5 with an inlet 10 for introducing air from the suction opening and an outlet 11, and includes a collection sensor mechanism 20 including the case 5, a signal processing unit 30 and a measuring unit 40.

In detection apparatus 100A, an air introducing mechanism 50 is provided. Air introducing mechanism 50 introduces air from the suction opening to case 5. Air introducing mechanism 50 may be a fan, a pump and their driving mechanism provided outside of case 5. It may, for example, be a heater, a micro-pump, a micro-fan and their driving mechanism built in case 5. Further, air introducing mechanism 50 may have a structure common to the air introducing mechanism of the air purifier portion of the air purifier. Preferably, the driving mechanism included in air introducing mechanism 50 is controlled by measuring unit 40 such that flow rate of introduced air is regulated. Preferably, the flow rate of air introduced by air introducing mechanism 50 is 1 L (liter)/min to 50 m³/min.

Collection sensor mechanism 20 includes a detecting mechanism, a collecting mechanism and a heating mechanism.

FIG. 2A shows as an example of the collecting mechanism a collecting mechanism including a discharge electrode 1, a collecting jig 12, and a high-voltage power supply 2. Discharge electrode 1 is electrically connected to a negative electrode of high-voltage power supply 2. The positive electrode of high-voltage power supply 2 is grounded. As a result, particles suspended in the introduced air are negatively charged near discharge electrode 1. Collecting jig 12 has a support board 4 formed, for example, of a glass plate, having a conductive, transparent coating 3. Coating 3 is grounded. Thus, the negatively charged particles suspended in the air move toward collecting jig 12 because of electrostatic force, and are attracted and held by conductive coating 3, whereby the particles are collected on collecting jig 12.

Support board 4 is not limited to a glass plate and it may be formed of ceramic, metal or other materials. Coating 3 formed on support board 4 is not limited to a transparent coating. As another example, support board 4 may include an insulating material such as ceramic, and a metal coating formed thereon. When support board 4 is of metal material, formation of a coating on its surface is unnecessary. More specifically, support board 4 can be a silicon board, a stainless used steel (SUS) board, a copper board, or the like.

The detecting mechanism includes: a light emitting element 6 as a light source; a lens (or lenses) 7, provided in the direction of light irradiation by emitting element 6, for collimating the light beams from light emitting element 6 or to adjust the light beams to a prescribed width; an aperture 8; a light receiving element 9; a collecting lens (or lenses) 13, provided in the direction of light reception by light receiving element 9, for collecting fluorescence generated by irradiation of airborne particles collected on collecting jig 12 by the collecting mechanism with light from light emitting element 6 to light receiving element 9; and a filter (or filters) 14 for preventing entrance of irradiating light beam to light receiving element 9. Aperture 8 is provided as needed. Conventional configurations may be applied to these components.

Light emitting element 6 may include a semiconductor laser 6 or an LED (Light

Emitting Diode) device. Wavelength of light may be in ultraviolet range or visible range, provided that the light can excite and cause fluorescent emission from particles of biological origin among the airborne particles. Preferable wavelength is 300 nm to 450 nm, with which tryptophan, NaDH, riboflavin and the like included in microorganisms and emitting fluorescence are efficiently excited, as disclosed in Japanese Patent Laying-Open No. 2008-508527. As light receiving element 9, conventional photo-diode, image sensor or the like is used.

Each of lens 7 and collecting lens 13 may be formed of plastic resin or glass. By a combination of lens 7 and aperture 8, light beams emitted from light emitting element 6 are collected on a surface of collecting jig 12, and form an irradiation region 15 on collecting jig 12. The shape of irradiation region 15 is not specifically limited, and it may have a circular, elliptical or rectangular shape. Though the size of irradiation region 15 is not specifically limited, preferably, the diameter of a circle, the longer side length of an ellipse or the length of one side of a rectangle is in the range of about 0.05 mm to 50 mm.

Filter 14 is formed of a single filer or a combination of different types of filters, and placed in front of collecting lens 13 or light receiving element 9. This prevents stray light derived from light emitted from light emitting element 6 and reflected by collecting jig 12 and case 5 from entering light receiving element 9 together with the fluorescence from particles collected by collecting jig 12.

The heating mechanism includes a heater 91 electrically connected to measuring unit 40 and having its amount of heating (heating time, heating temperature) controlled by measuring unit 40. Suitable heater 91 includes a ceramic heater. While in the following description, heater 91 is assumed as a ceramic heater, it may be a different heater, such as an infrared heater, an infrared lamp, or the like.

Heater 91 is provided at a position that can heat the airborne particles collected on collecting jig 12 and separated by some means or other at least at the time of heating from sensor equipment including light emitting element 6 and light receiving element 9. Preferably, as shown in FIG. 2A, the heater is arranged on a side away from the sensor equipment such as light emitting element 6 and light receiving element 9, with collecting jig 12 placed in between. By such an arrangement, at the time of heating, heater 91 is separated by collecting jig 12 from the sensor equipment including light emitting element 6 and light receiving element 9, whereby influence of heat on light emitting element 6, light receiving element 9 and the like can be prevented. More preferably, heater 91 is surrounded by heat insulating material as shown in FIG. 2B. Suitable heat insulating material includes glass epoxy resin. With such a structure, the inventors confirmed that when heater 91 implemented by a ceramic heater reached 200° C. in about 2 minutes, the temperature of a portion (not shown) connected to heater 91 with the heat insulating member interposed was not higher than 30° C.

Case 5 has a rectangular parallelepiped shape with the length of each side being 3 mm to 500 mm. Though case 5 has a rectangular parallelepiped shape in the present embodiment, the shape is not limited, and the case may have a different shape. Preferably, at least the inner side is painted black or treated with black alumite. This prevents reflection of light from the inner wall surface as a cause of stray light. Though the material of case 5 is not specifically limited, preferably, plastic resin, aluminum, stainless steel or a combination of these may be used. Inlet 10 and outlet 11 of case 5 have circular shape with the diameter of 1 mm to 50 mm. The shape of inlet 10 and outlet 11 is not limited to a circle, and it may be an ellipse or a rectangle.

As described above, filter 14 is placed in front of light receiving element 9 and serves to prevent entrance of stray light to light receiving element 9. In order to attain higher fluorescent intensity, however, it becomes necessary to increase intensity of light emitted from light emitting element 6. This leads to higher intensity of reflected light, that is, increased intensity of stray light. Therefore, light emitting element 6 and light receiving element 9 are arranged to have such a positional relation that the stray light intensity is kept lower than the light intercepting effect attained by filter 14.

An exemplary arrangement of light emitting element 6 and light receiving element 9 will be described with reference to FIGS. 2A, 3A and 3B. FIG. 3A is a cross-sectional view of detection apparatus 100A viewed from the position of IIIA-IIIA of FIG. 2A in the direction of the arrow, and FIG. 3B is a cross-sectional view taken from the position of IIIB-IIIB of FIG. 3A in the direction of the arrow. For convenience of description, in these figures, collecting mechanism other than collecting jig 12 is not shown.

Referring to FIG. 3A, when viewed from the direction of arrow IIIA (top surface) of FIG. 2A, light emitting element 6 and lens 7 are arranged at a right angle or approximately at a right angle to light receiving element 9 and collecting lens 13. The light from light emitting element 6, passing through lens 7 and aperture 8 and reflected from irradiation region 15 formed on the surface of collecting jig 12 proceeds in the direction along the incident light. Therefore, by such a structure, direct entrance of the reflected light to light receiving element 9 is avoided. The fluorescence emitted from the surface of collecting jig 12 is isotropic and, therefore, the arrangement is not limited to the above as long as the entrance of reflected light and stray light to light receiving element 9 can be prevented.

More preferably, collecting jig 12 is provided with a configuration for collecting fluorescence emitted from particles trapped on the surface corresponding to irradiation region 15 to light receiving element 9. Such a configuration corresponds, for example, to a spherical recess 51 shown in FIG. 3B. Further, preferably, collecting jig 12 is provided inclined by an angle Theta in a direction to light receiving element 9 so that the surface of collecting jig 12 faces light receiving element 9. By such a configuration, the fluorescence isotropically emitted from the particles in spherical recess 51 is reflected on the spherical surface and effectively collected in the direction to light receiving element 9, whereby the light receiving signal can be intensified. Though the size of recess 51 is not limited, preferably, it is made larger than irradiation region 15.

Again referring to FIG. 2A, light receiving element 9 is connected to signal processing unit 30 and outputs a current signal in proportion to the intensity of received light to signal processing unit 30. Therefore, fluorescence emitted from the particles that have been suspended in the introduced air, collected to the surface of collecting jig and irradiated with light from light emitting element 6, is received by light receiving element 9 and the intensity of received light is detected by signal processing unit 30.

Further, inlet 10 and outlet 11 of case 5 are provided with shutters 16A and 16B, respectively. Shutters 16A and 16B are connected to measuring unit 40 and have their opening/closing controlled. When shutters 16A and 16B are closed, air flow and entrance of external light to case 5 are blocked. Measuring unit 40 closes shutters 16A and 16B at the time of fluorescence measurement as will be described later, to block air flow and entrance of external light to case 5. Consequently, at the time of fluorescence measurement, collection of airborne particles by the collecting mechanism is stopped. Further, since entrance of external light to case 5 is blocked, stray light in case 5 can be reduced. Provision of only one of shutters 16A and 16B, for example, only shutter 16B on the side of outlet 11 may suffice.

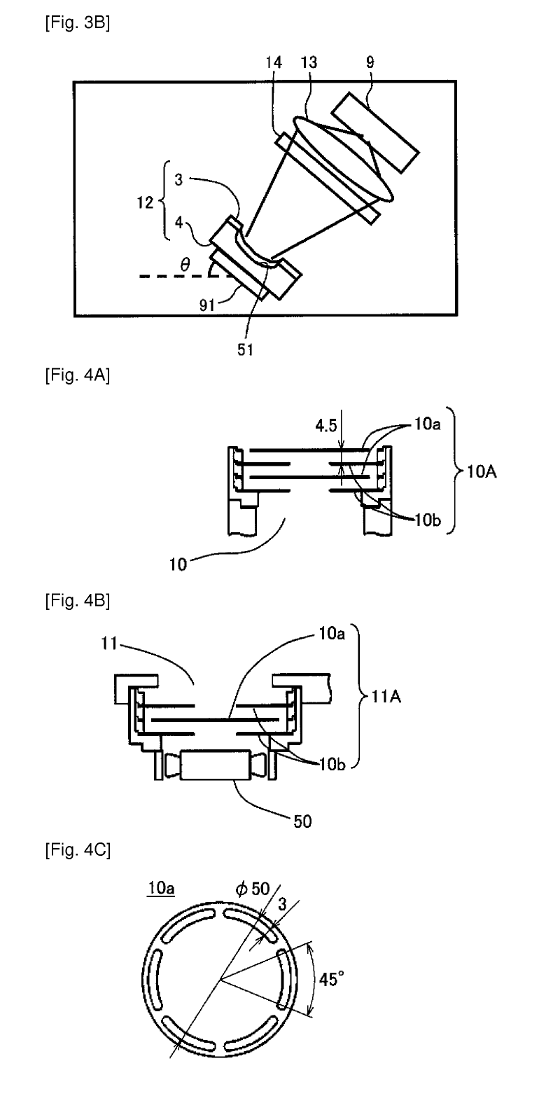

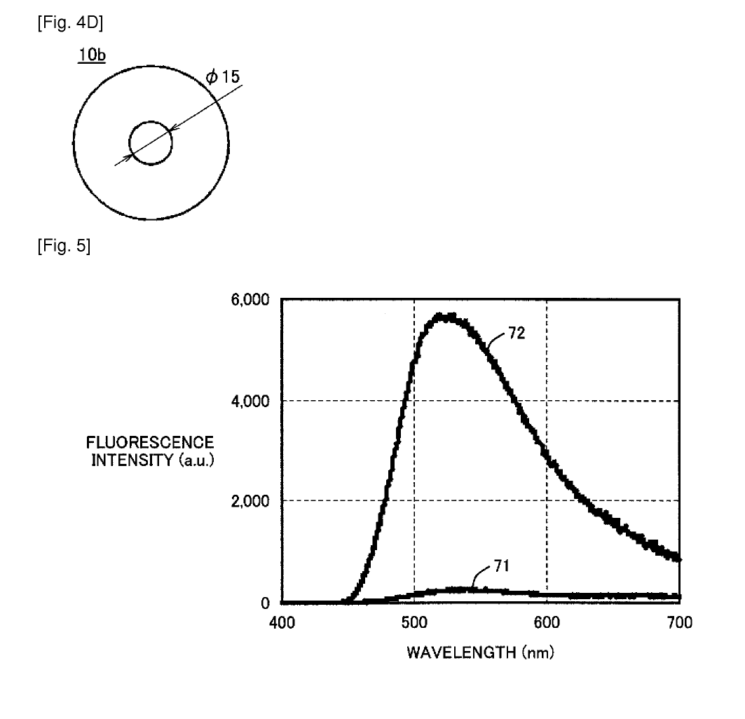

Further, as a configuration allowing air flow to/from case 5 but intercepting entrance of external light, light shielding portions 10A and 11A such as shown in FIGS. 4A and 4B, may be provided on inlet 10 and outlet 11.

Referring to FIGS. 4A and 4B, light shielding portions 10A and 11A provided on inlet 10 and outlet 11 both have light shielding plates 10a and 10b overlapped alternately at an interval of about 4.5 mm. Light shielding plates 10a and 10b have holes formed therein at portions not overlapping with each other, with the shape of holes corresponding to the shape of inlet 10 and outlet 11 (here, circular shape), such as shown in FIGS. 4C and 4D, respectively. Specifically, light shielding plate 10a has holes opened at the circumferential portions, and light shielding plate 10b has a hole opened at the center. When light shielding plates 10a and 10b are overlapped, the holes formed in respective plates do not overlap. As shown in FIG. 4A, in light shielding portion 10A for inlet 10, light shielding plate 10a, light shielding plate 10b, light shielding plate 10a and light shielding plate 10b are arranged in this order from the outer side to the inner side. As shown in FIG. 4B, in light shielding portion 11A for outlet 11, light shielding plate 10b, light shielding plate 10a and light shielding plate 10b are arranged in this order from the outside (on the side of air introducing mechanism 50) to the inside. By this configuration, though air flow to/from case 5 is possible, entrance of external light is intercepted, and stray light in case 5 can be reduced.

Signal processing unit 30 is connected to measuring unit 40 and outputs a result of current signal processing to measuring unit 40. Based on the result of processing from signal processing unit 30, measuring unit 40 performs a process for displaying the result of measurement on display panel 130.

The detection apparatus according to the present embodiment detects an amount of airborne particles of biological origin. While “particles of biological origin” as referred to in the following description are typically represented by microbes and other microorganisms (including their corpses), they also include any other biological entity that performs biotic activity or a portion of the biological entity, that has a size allowing the biological entity or a portion thereof to be airborne, regardless of whether it may be dead or alive. More specifically, other than microbes and other microorganisms (including their corpses), the particles of biological origin can also include pollen, mites (including their corpses), and the like. In the following description, “microorganisms” will represent “particles of biological origin”, and pollen and the like will also be considered similarly.

Here, the principle of detection in the detection apparatus will be described.

As disclosed in Japanese Patent Laying-Open No. 2008-508527, it has been conventionally known that when airborne particles of biological origin are irradiated with ultraviolet or blue light, the particles emit fluorescence. In the air, however, other particles that emit fluorescence such as dust and lint of chemical fiber are also suspended. Therefore, it is impossible by simply detecting fluorescence to distinguish whether the light comes from particles of biological origin or from, for example, dust of chemical fiber.

In view of the foregoing, the inventors conducted heat treatment on particles of biological origin and on dust of chemical fiber and the like, and measured changes in fluorescence before and after heating. FIGS. 5 to 14 show specific results of measurement by the inventors. From the measurement results, the inventors found that the fluorescence intensity from dust did not change before and after heating, while fluorescence intensity emitted from biological particles increased after heating.

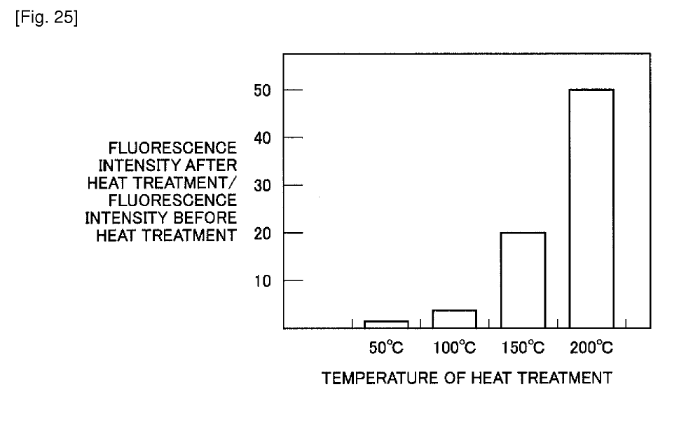

Furthermore, the present inventors subjected Penicillium to a heat treatment at different temperatures for five minutes and measured a ratio of intensity of fluorescence provided from Penicillium before and after the heat treatment (i.e., intensity of fluorescence after the heat treatment/intensity of fluorescence before the heat treatment). FIG. 25 shows a relationship between temperature of a heat treatment of Penicillium and a ratio of intensity of fluorescence provided from Penicillium before and after the heat treatment, as obtained from the measurement done by the present inventors. From the measurement, it has been found that, as shown in FIG. 25, when Penicillium was heated at 50° C., its fluorescence intensity hardly varied between before and after it was heated, and that when it was heated at 100° C. or higher, its fluorescence intensity significantly increased. Furthermore, although not shown in the figure, it has also been found that when it was heated at 250° C., its fluorescence intensity varied less than when it was heated at 200° C. From this measurement, the present inventors have found that a heat treatment of 100° C. to 250° C. is suitable, and more preferably, a heat treatment of 200° C. is more suitable. Accordingly, the present inventors subjected a variety of specimens to a heat treatment at 200° C. for five minutes and thus measured how the fluorescence from each specimen varies between before and after the heat treatment.

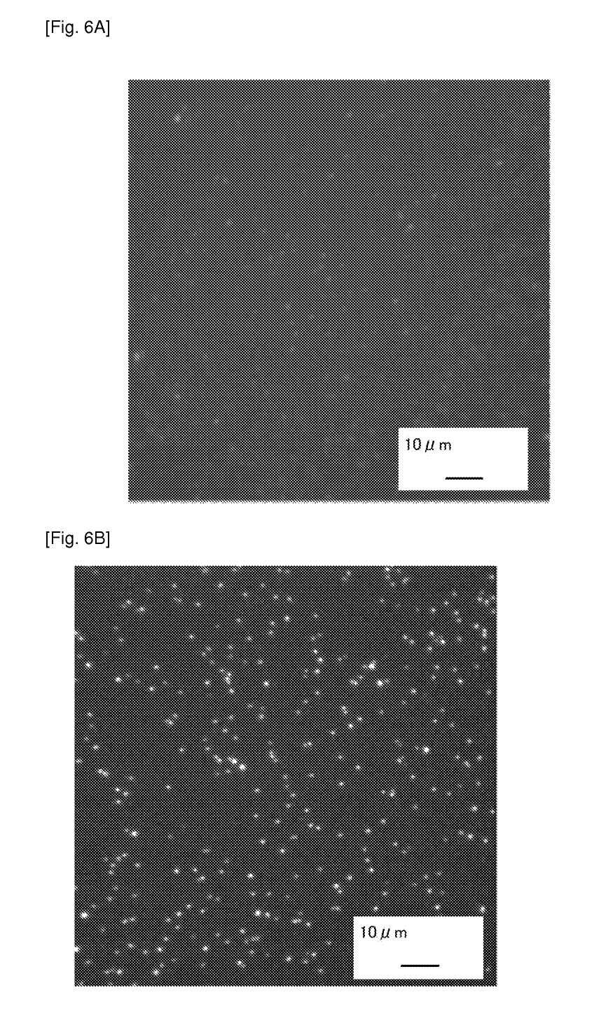

More specifically, FIG. 5 shows results of measurement of fluorescent spectra before (curve 71) and after (curve 72) heat treatment of Escherichia coli as biological particles at 200° C. for 5 minutes. From the results of measurement shown in FIG. 5, it can be seen that the fluorescence intensity from Escherichia coli increased significantly by the heat treatment. It is also apparent from the comparison between a fluorescent micrograph of Escherichia coli before heat treatment of FIG. 6A and a fluorescent micrograph of Escherichia coli after heat treatment of FIG. 6B that the fluorescence intensity from Escherichia coli increased significantly by the heat treatment.

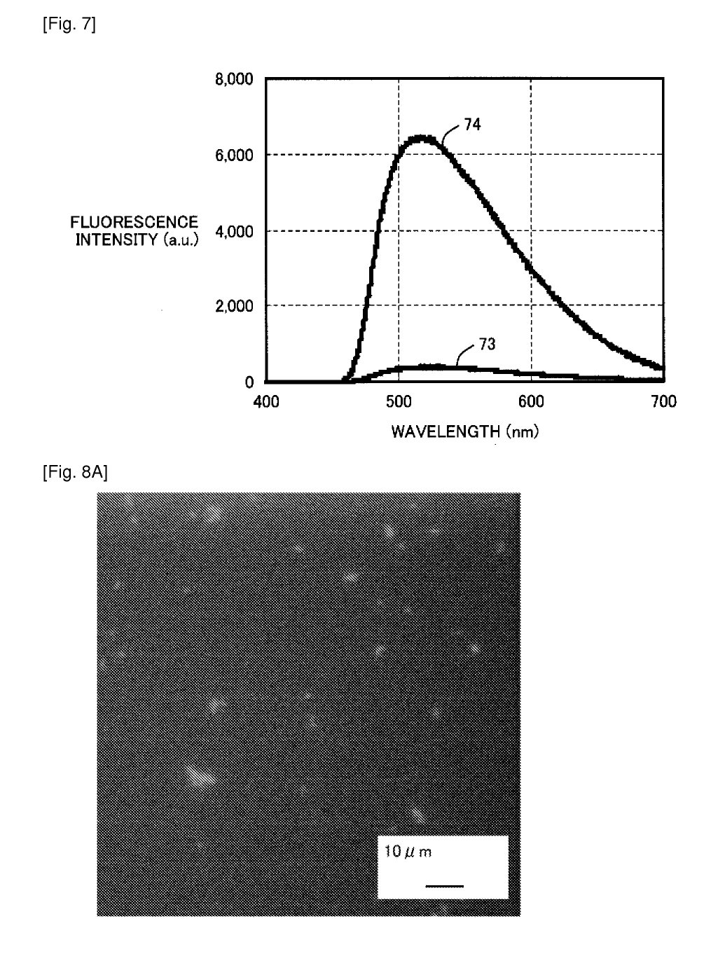

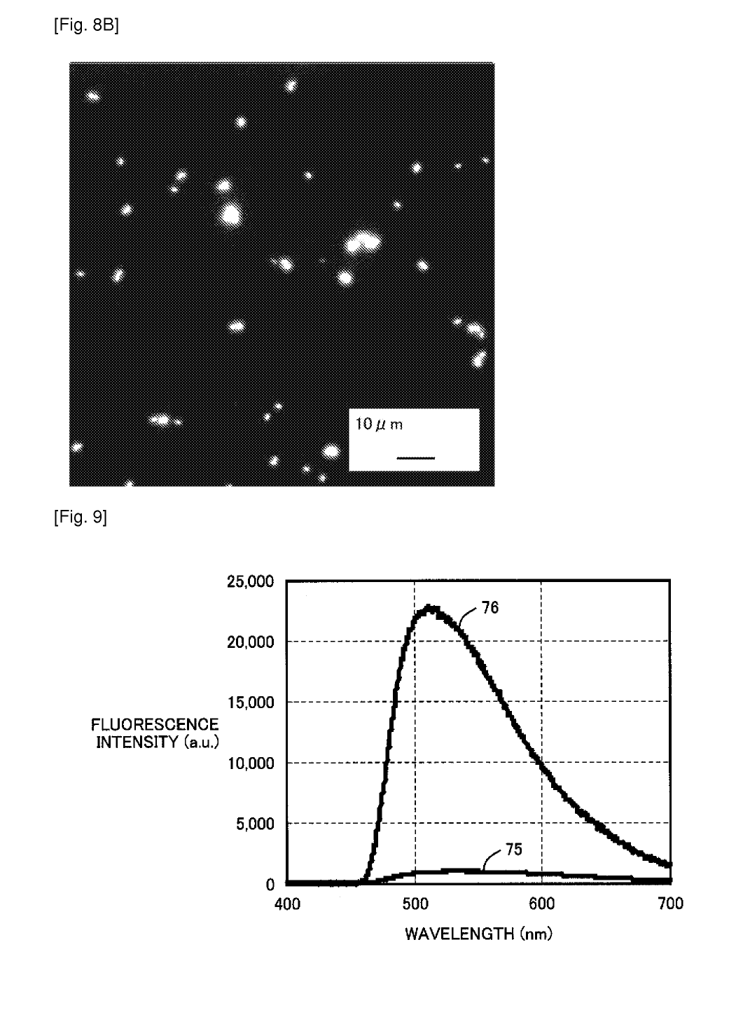

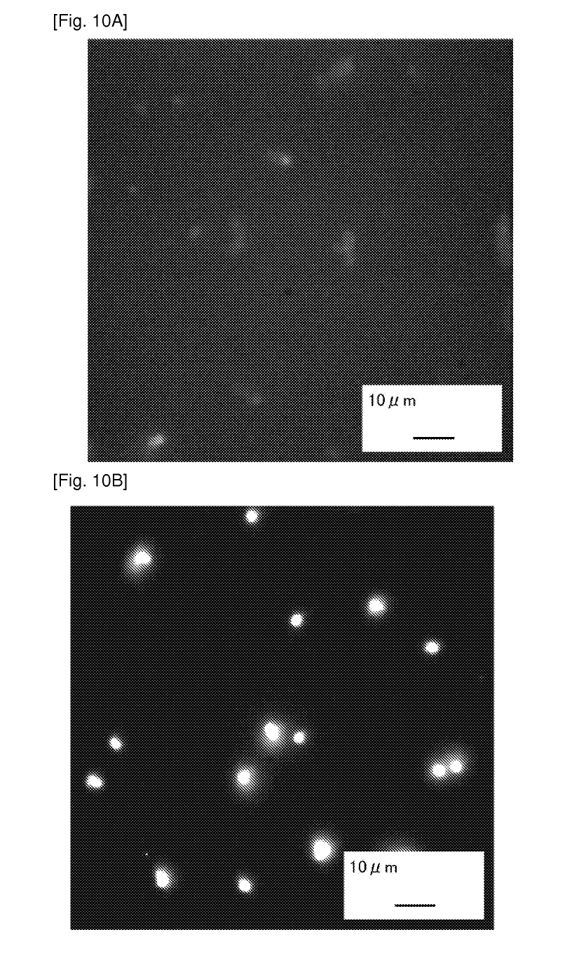

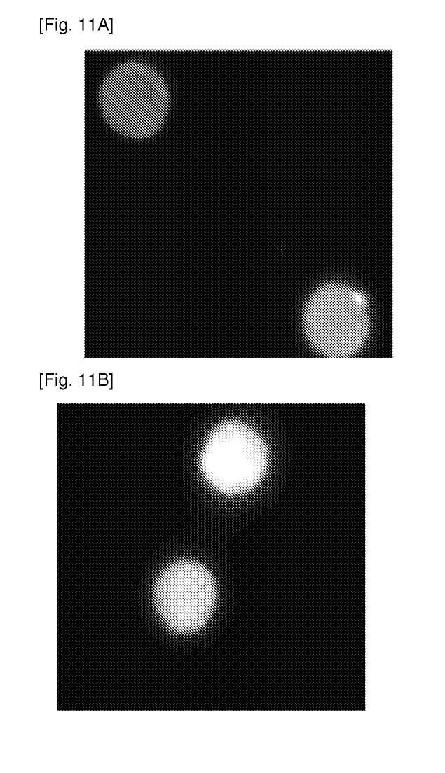

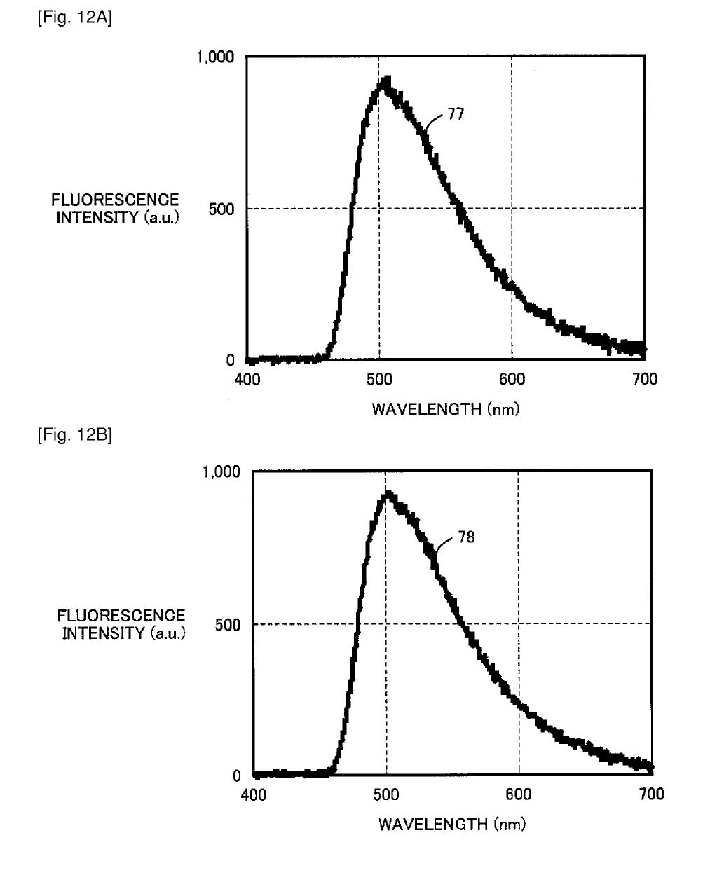

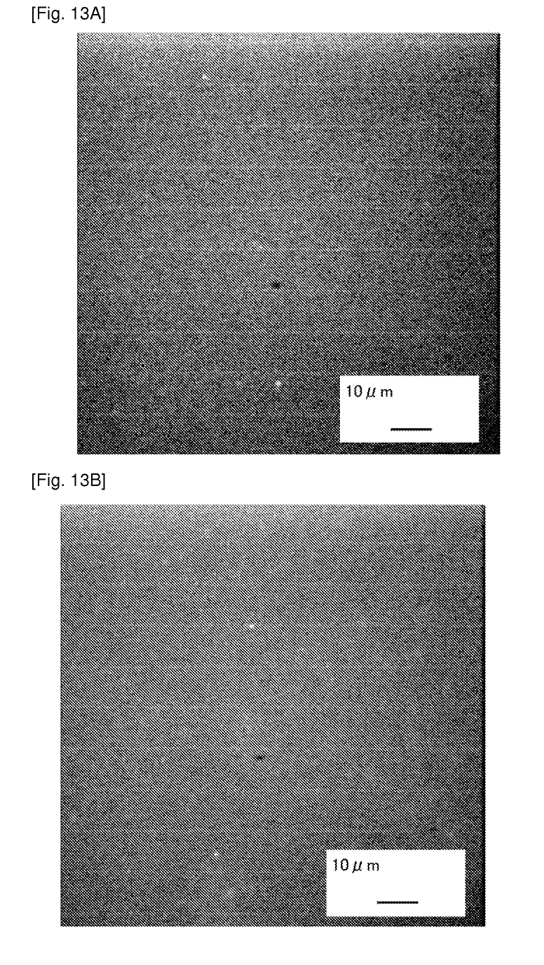

Similarly, FIG. 7 shows results of measurement of fluorescent spectra before (curve 73) and after (curve 74) heat treatment of Bacillius subtilis as biological particles at 200° C. for 5 minutes, and FIG. 8A is a fluorescent micrograph before heat treatment and FIG. 8B is a fluorescent micrograph after heat treatment. FIG. 9 shows results of measurement of fluorescent spectra before (curve 75) and after (curve 76) heat treatment of Penicillium as biological particles at 200° C. for 5 minutes, and FIG. 10A is a fluorescent micrograph before heat treatment and FIG. 10B is a fluorescent micrograph after heat treatment. Furthermore, FIGS. 11A and 11B are fluorescent micrographs of cedar pollen as particles of biological origin before and after heat treatment, respectively, at 200° C. for five minutes. As can be seen from these results, as in the case of Escherichia coli, the fluorescence intensity from particles of a different biological origin is also increased significantly by the heat treatment.

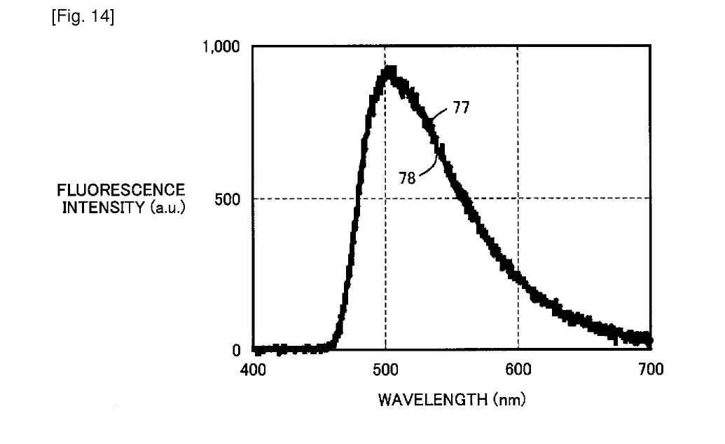

In contrast, FIGS. 12A and 12B show results of measurement of fluorescent spectra before (curve 77) and after (curve 78) heat treatment of fluorescence-emitting dust at 200° C. for 5 minutes, and FIG. 13A is a fluorescent micrograph before heat treatment and FIG. 13B is a fluorescent micrograph after heat treatment. Placing the fluorescent spectrum of FIG. 12A on the fluorescent spectrum of FIG. 12B, we obtain FIG. 14, from which it can be verified that these spectra substantially overlap with each other. Specifically, from the result of FIG. 14 and from the comparison between FIGS. 13A and 13B, it can be seen that the fluorescence intensity from dust does not change before and after heat treatment.

As the principle of detection in detection apparatus 100, the above-described phenomenon verified by the inventors is applied. Specifically, dust, dust with biological particles adhered, and particles of biological origin are suspended in the air. From the phenomenon described above, it follows that if collected particles include fluorescence-emitting dust, the fluorescent spectra measured before heat treatment include fluorescence from particles of biological origin and fluorescence from fluorescence-emitting dust and, therefore, it is impossible to distinguish particles of biological origin from, for example, dust of chemical fiber. By the heat treatment, however, the fluorescence intensity from only the particles of biological origin increases, while the fluorescence intensity from fluorescence-emitting dust does not change. Therefore, by measuring the difference of fluorescence intensity before heat treatment and fluorescence intensity after prescribed heat treatment, it is possible to find the amount of particles of biological origin.

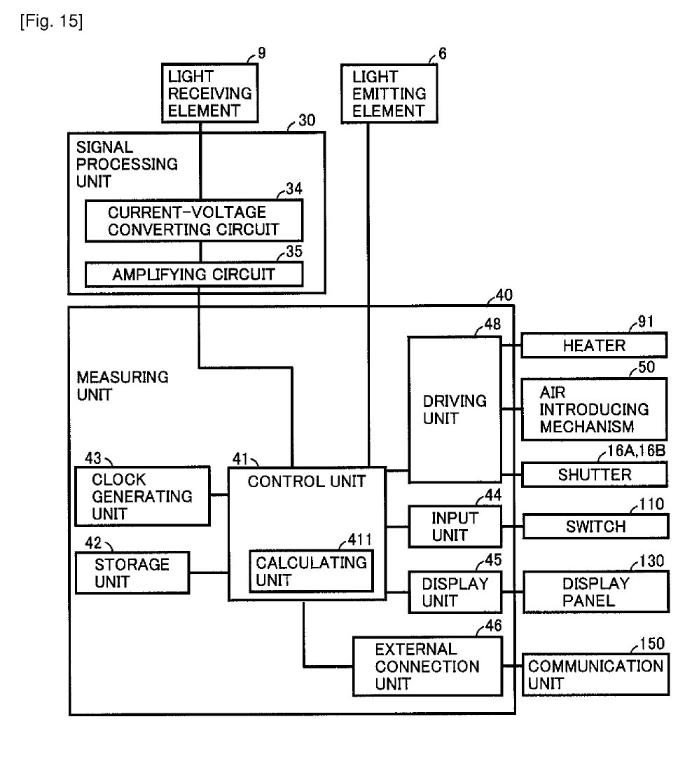

The functional configuration of detection apparatus 100A for detecting airborne microorganisms utilizing the principle will be described with reference to FIG. 15. FIG. 15 shows an example in which the functions of signal processing unit 30 are implemented by hardware configuration mainly of electric circuitry. It is noted, however, that at least part of the functions may be implemented by software configuration realized by a CPU (Central Processing Unit), not shown, provided in signal processing unit 30, executing a prescribed program. Further, in the example shown, measuring unit 40 is implemented by software configuration. At least part of the functions thereof may be realized by hardware configuration such as electric circuitry.

Referring to FIG. 15, signal processing unit 30 includes a current-voltage converting circuit 34 connected to light receiving element 9, and an amplifying circuit 35 connected to current-voltage converting circuit 34.

Measuring unit 40 includes a control unit 41, a storage unit 42, and a clock generating unit 43. Further, measuring unit 40 includes: an input unit 44 for receiving input of information by receiving an input signal from switch 110 upon operation of switch 110; a display unit 45 executing a process for displaying results of measurement and the like on display panel 130; an external connection unit 46 performing processes required for exchanging data and the like with an external apparatus connected to communication unit 150; and a driving unit 48 for driving shutters 16A and 16B, air introducing mechanism 50 and heater 91.

When particles introduced to case 5 and collected on collecting jig 12 are irradiated with light from light emitting element 6, fluorescence emitted from the particles in the irradiation region is collected at light receiving element 9. Light receiving element 9 outputs a current signal in accordance with the amount of received light to signal processing unit 30. The current signal is input to current-voltage converting circuit 34.

Current-voltage converting circuit 34 detects a peak current value H representing the fluorescence intensity from the current signal input from light receiving element 9, and converts it to a voltage value Eh. The voltage value Eh is amplified by amplifying circuit 35 by a preset gain, and the result is output to measuring unit 40. Control unit 41 of measuring unit 40 receives the input of voltage value Eh from signal processing unit 30 and successively stores in storage unit 42.

Clock generating unit 43 generates and outputs clock signals to control unit 41. With the timing based on the clock signals, control unit 41 outputs control signals for opening and closing shutters 16A and 16B to driving unit 48, to control opening/closing of shutters 16A and 16B. Further, control unit 41 is electrically connected to light emitting element 6 and light receiving element 9, and controls ON/OFF of these elements.

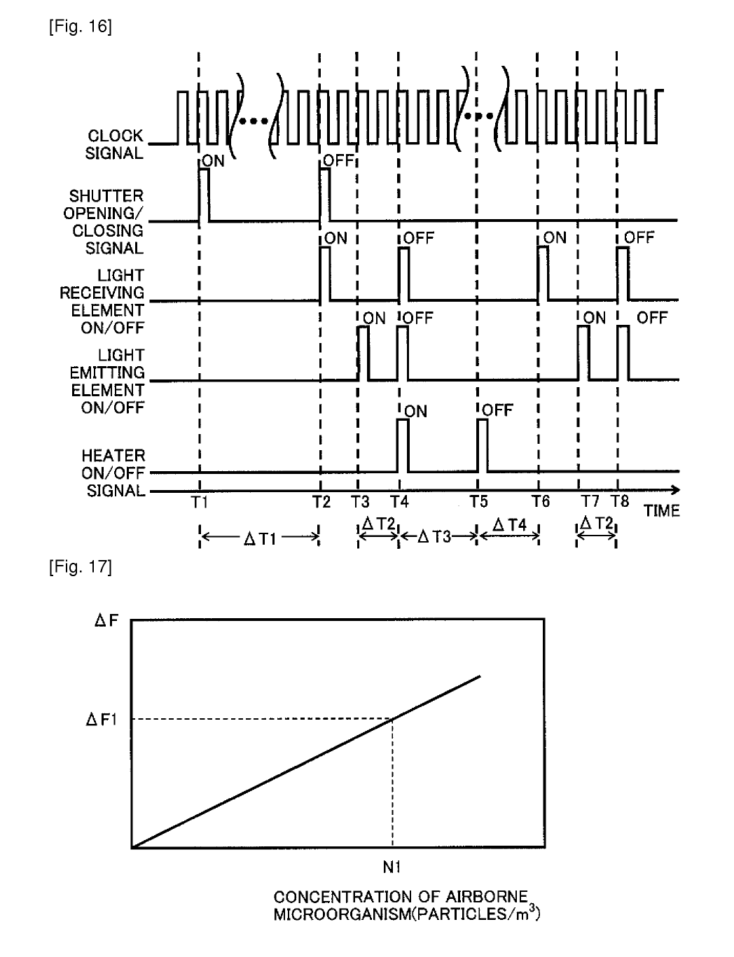

Control unit 41 includes a calculating unit 411. Calculating unit 411 operates to calculate the amount of particles of biological origin suspended in the introduced air, using the voltage value Eh stored in storage unit 42. Specific operation will be described using a time chart of FIG. 16, showing the flow of control by control unit 41. Here, as the amount of particles of biological origin, it is assumed that concentration of microorganisms suspended in the air introduced to case 5 is calculated.

Referring to FIG. 16, when detection apparatus 100A is powered ON, control unit 41 of measuring unit 40 outputs a control signal to driving unit 48, to drive air introducing mechanism 50. Further, at a time point T1 based on the clock signal from clock generating unit 43, control unit 41 outputs a control signal for opening (ON) shutters 16A and 16B to driving unit 48. Then, at time point T2 after the lapse of DeltaT1 from T1, control unit 41 outputs a control signal for closing (OFF) shutters 16A and 16B to driving unit 48.

Thus, for the time period DeltaT1 from T1, shutters 16A and 16B are opened, and as air introducing mechanism is driven, external air is introduced through inlet 10 to case 5. Particles suspended in the air introduced to case 5 are negatively charged by discharge electrode 1, and by the air flow and an electric field formed between discharge electrode 1 and coating 3 on the surface of collecting jig 12, the particles are collected on the surface of collecting jig 12 for the time period DeltaT1.

At time point T2, shutters 16A and 16B are closed, so that the air flow in case 5 stops. Thus, collection of airborne particles by collecting jig 12 ends. Further, stray light from the outside is blocked.

At time point T2 when shutters 16A and 16B are closed, control unit 41 outputs a control signal to light receiving element 9 to start reception of light (ON). At the same time (T2) or at T3 slightly after T2, it outputs a control signal to light emitting element 6 to start emission of light (ON). Thereafter, at time point T4 after the lapse of DeltaT2, which is a predefined measurement time for measuring fluorescence intensity, from time T3, control unit 41 outputs a control signal to light receiving element 9 to stop reception of light (OFF) and a control signal to light emitting element 6 to stop emission of light (OFF). The measurement time may be set in advance in control unit 41, or it may be input or changed by an operation of, for example, switch 110, by a signal from PC 300 connected to communication unit 150 through cable 400, or by a signal from a recording medium attached to communication unit 150.

Specifically, from time point T3 (or from T2), emission of light from light emitting element 6 starts. The light from light emitting element 6 is directed to irradiation region 15 on the surface of collecting jig 12, and fluorescence is emitted from collected particles. Fluorescence is received by light receiving element 9 for the defined measuring time DeltaT2 from time T3, and a voltage value in accordance with the fluorescence intensity F1 is input to measuring unit 40 and stored in storage unit 42.

At this time, a separate light emitting element such as an LED (not shown) may be provided, light emitted from this element and reflected from a reflection region (not shown), at which particles are not collected, on the surface of collecting jig 12 may be collected by a separate light receiving element (not shown), the intensity of received light may be used as a reference value I0 and the value F1/I0 may be stored in storage unit 42. By calculating the ratio to reference value I0, it becomes advantageously possible to compensate for the fluctuation of fluorescence intensity derived from environmental conditions such as moisture and temperature of light emitting element or light receiving element, or from variation in characteristics caused by deterioration or aging.

At time point T4 (or a time point slightly later than T4) when emission of light by light emitting element 6 and reception of light by light receiving element 9 are stopped, control unit 41 outputs a control signal to heater 91 to start heating (ON). Thereafter, at time point T5 after the lapse of DeltaT3, which is a predefined heating time for the heat treatment, from the start of heating by heater 91 (from time point T4 or a time point slightly later than T4), control unit 41 outputs a control signal to heater 91 to stop heating (OFF).

Thus, for the time period DeltaT3 of heating from T4 (or a time point slightly later than T4), heat treatment is done on the particles collected in irradiation region 15 on the surface of collecting jig 12, by heater 91. The heating temperature at this time is defined in advance. By the heat treatment for the time period DeltaT3, the particles collected on the surface of collecting jig 12 are heated by prescribed heat inputs. As in the case of the measurement time described above, the time of heat treatment DeltaT3 (that is, the heat input) may be set in advance in control unit 41, or it may be input or changed by an operation of, for example, switch 110, by a signal from PC 300 connected to communication unit 150 through cable 400, or by a signal from a recording medium attached to communication unit 150.

Thereafter, for a time period DeltaT4, the heated particles are subjected to cooling. For the cooling process, air introducing mechanism 50 may be used. In that case, external air may be taken in from an opening (not shown in FIG. 2) provided with an HEPA (High Efficiency Particulate Air) filter. Alternatively, a separate cooling mechanism such as a Peltier device may be used.

Thereafter, control unit 41 outputs a control signal to end the operation of air introducing mechanism 50, and at time T6, outputs a control signal to light receiving element 9 to start reception of light (ON). At the same time (T6) or at time T7 slightly later than T6, it outputs a control signal to light emitting element 6 to start emission of light (ON). Thereafter at time point T8 after the lapse of DeltaT2 from T7, control unit 41 outputs a control signal to light receiving unit 9 to stop reception of light (OFF) and a control signal to light emitting element 6 to stop emission of light (OFF).

In this manner, after heat treatment for the time period DeltaT3, from the particles collected in irradiation region 15 on the surface of collecting jig 12 irradiated by light emitting element 6, the fluorescence for the measurement time DeltaT2 is received by light receiving element 9. The voltage value corresponding to the fluorescence intensity F2 is input to measuring unit 40 and stored in storage unit 42.

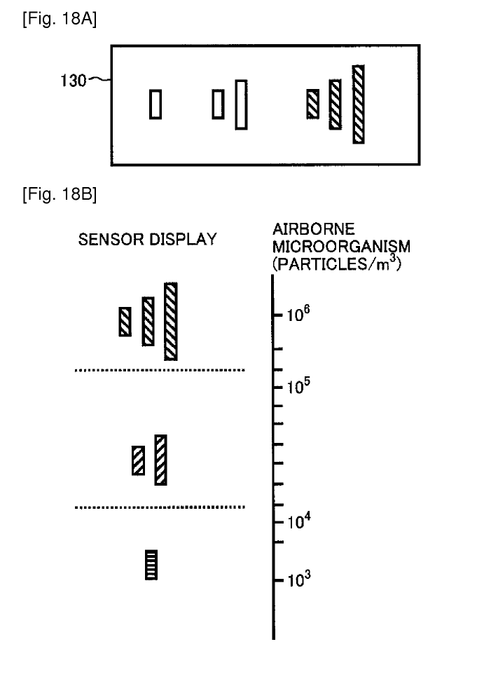

Calculating unit 411 calculates a difference between the stored fluorescence intensity F1 and fluorescence intensity F2 as an amount of increase DeltaF. As described above, the amount of increase DeltaF relates to the amount of biological particles (the number or concentration of particles). Calculating unit 411 stores beforehand the correspondence between the amount of increase DeltaF and the amount of biological particles (the concentration of particles) such as shown in FIG. 17. Then, calculating unit 411 provides the concentration of particles of biological origin, obtained by using the amount of increase DeltaF and the correspondence relation, as the concentration of particles of biological origin in the air introduced to case 5 in time period DeltaT1.

The correspondence relation between the amount of increase DeltaF and the concentration of particles of biological origin is experimentally determined in advance. By way of example, one type of microorganism such as Escherichia coli, Bacillius subtilis or Penicillium is sprayed using a nebulizer in a vessel having the size of 1 m³. While the concentration of microorganisms is kept at N (particles/m³), the microorganisms are collected using detection apparatus 100 by the method of detection described above for the time period DeltaT1. Then, the collected microorganisms are heated by a prescribed heat input (heating time DeltaT3, prescribed heating temperature) using heater 91, cooled for a prescribed time period DeltaT4, and the amount of increase DeltaF of fluorescence intensity before and after heating is measured. Similar measurements are made for various concentrations of microorganisms, whereby the relation between the amount of increase DeltaF and the microorganism concentration (particles/m³) can be found as shown in FIG. 17.

The correspondence relation between the amount of increase DeltaF and the concentration of biological particles may be input by an operation of switch 110 or the like and stored in calculation unit 411. Alternatively, a recording medium having the correspondence relation recorded thereon may be attached to communication unit 150 and read by external connection unit 46 and stored in calculation unit 411. It may be input and transmitted by PC 300, received by external connection unit 46 through cable 400 connected to communication unit 150, and stored in calculation unit 411. If communication unit 150 is adapted to infrared or Internet communication, the correspondence relation may be received by external connection unit 46 at communication unit 150 by such communication, and stored in calculation unit 411. Further, the correspondence relation once stored in calculation unit 411 may be updated by measuring unit 40.

If the amount of increase DeltaF is calculated to be a difference DeltaF1, calculation unit 411 identifies a value corresponding to the increased amount DeltaF1 from the correspondence relation shown in FIG. 17, and thereby calculates the concentration N1 (particles/m³) of particles of biological origin.

It is noted, however, that the correspondence relation between the amount of increase DeltaF and the microorganism concentration possibly differs depending on the type of microorganism (for examples, types of microbes). Therefore, calculation unit 411 defines some microorganism as standard microorganism and stores the correspondence relation between the amount of increase DeltaF and the microorganism concentration. In this manner, microorganism concentration in various environments can be calculated as the microorganism concentration in equivalence of the standard microorganism, whereby environmental management becomes easier.

Though the difference in fluorescence intensity before and after heat treatment of a prescribed heat input (prescribed heating temperature, heating time DeltaT3) is used as the amount of increase DeltaF in the embodiment above, the ratio thereof may be used.

The concentration of biological particles or microorganisms among the collected particles calculated by calculation unit 411 is output from control unit 41 to display unit 45. Display unit 45 performs a process for displaying the input microorganism concentration on display unit 130. An example of the display on display panel 130 is a sensor display of FIG. 18A. Specifically, on display panel 130, lamps corresponding to concentrations are provided, and display unit 45 specifies a lamp corresponding to the calculated concentration and lights the lamp as shown in FIG. 18B. As another example, it is also possible to light the lamp in different color in accordance with the calculated concentration. The display on display panel 130 is not limited to lamps, and numerical values or concentrations or messages prepared beforehand for corresponding concentrations may be displayed. The results of measurement may be written to a recording medium attached to communication unit 150, or may be transmitted to PC 300 through cable 400 connected to communication unit 150.

Input unit 44 may receive selection of the display method on display panel 130 in accordance with an operation signal from switch 110. Selection may be made possible as to whether the measurement results are to be displayed on display panel 130 or output to an external apparatus. A signal indicating the contents of selection may be output to control unit 41, and then a necessary control signal is output from control unit 41 to display unit 45 and/or external connection unit 46.

In this manner, detection apparatus 100A utilizes difference in characteristics when heated between the fluorescence from particles of biological origin and the fluorescence from fluorescence-emitting dust, and based on the amount of increase after a prescribed heat treatment, particles of biological origin are detected. Specifically, detection apparatus 100A detects the particles of biological origin utilizing the phenomenon that when the collected biological particles and dust are subjected to heat treatment, the fluorescence intensity from microorganisms increases whereas the fluorescence intensity from dust does not change. Therefore, even if fluorescence-emitting dust is suspended in the introduced air, it is possible to detect biological particles separate from fluorescence-emitting dust on real-time basis with high accuracy.

Further, detection apparatus 100A is controlled in the manner as shown in FIG. 16 and thereby shutters 16A and 16B are closed at the transition from the collecting step by the collecting mechanism to the detection step by the detecting mechanism. As a result, stray light caused by scattering at airborne particles during fluorescence measurement can be reduced and measurement accuracy can be improved.

### Second Embodiment

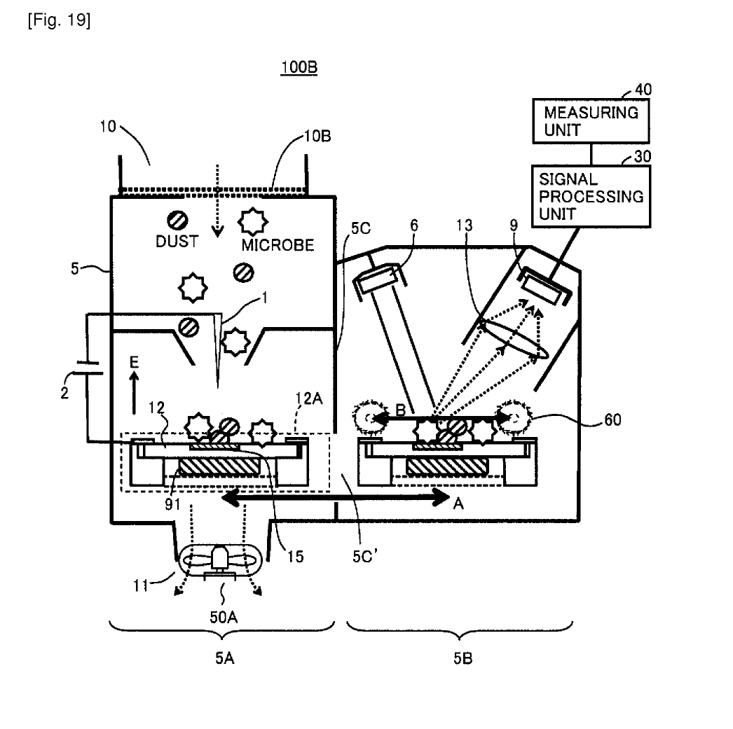

As shown in FIG. 19, a detection apparatus 100B in accordance with the second embodiment includes a detecting mechanism, a collecting mechanism and a heating mechanism. In FIG. 19, members denoted by the same reference characters as in detection apparatus 100A are substantially the same as the corresponding members of detection apparatus 100A. In the following, the difference over detection apparatus 100A will be mainly described.

More specifically, referring to FIG. 19, detection apparatus 100B is provided with a collection chamber 5A including at least a part of the collecting mechanism, and a detection chamber 5B including the detecting mechanism, sectioned by a partition wall 5C having a hole 5C′. In collection chamber 5A, a needle-shaped discharge electrode 1 and collecting jig 12 as the collecting mechanism are provided, and in detection chamber 5B, light emitting element 6, light receiving element 9 and collecting lens 13 as the detecting mechanism are provided.

On the side of discharge electrode 1 and collecting jig 12 of collection chamber 5A, inlet 10 and outlet 11 are provided, respectively, for introducing air to collection chamber 5A. Further, as shown in FIG. 19, a filter (pre-filter) 10B may be provided at inlet 10.

Inlet 10 and outlet 11 may be provided with light shielding portions 10A and 10B such as shown in FIGS. 4A and 4B similar to those of detection apparatus 100A, for intercepting entrance of external light while allowing air flow to/from collection chamber 5A.

A fan 50A as the air introducing mechanism is provided close to outlet 11. By fan 50A, the air is introduced from the inlet to collection chamber 5A. Air introducing mechanism 50 may be a pump and its driving mechanism provided outside of collection chamber 5A. It may, for example, be a heater, a micro-pump, a micro-fan and their driving mechanism built in collection chamber 5A. Further, fan 50A may have a structure common to the air introducing mechanism of the air purifier portion of the air purifier. Preferably, the driving mechanism of fan 50A is controlled by measuring unit 40 such that flow rate of introduced air is regulated. Preferably, the flow rate of air introduced by fan 50A is 1 L (liter)/min to 50 m³/min. When driven by a driving mechanism, not shown, controlled by measuring unit 40, fan 50A introduces air outside collection chamber 5A through inlet 10 and discharges air in collection chamber 5A through outlet 11 to the outside of collection chamber 5A as shown by a dotted line arrow in FIG. 19.

As the collecting mechanism, a collecting mechanism similar to that of detection apparatus 100A may be used. Specifically, referring to FIG. 19, the collecting mechanism includes discharge electrode 1, collecting jig 12, and high-voltage power supply 2. Discharge electrode 1 is electrically connected to the positive electrode of high-voltage power supply 2. Collecting jig 12 is electrically connected to a negative electrode of high-voltage power supply 2.

Collecting jig 12 is a support board formed, for example, of a glass plate, having a conductive, transparent coating, as in detection apparatus 100A. The coating side of collecting jig 12 is electrically connected to the negative electrode of high-voltage power supply 2. Thus, there is generated a potential difference between discharge electrode 1 and collecting jig 12, and an electric field in the direction indicated by an arrow E of FIG. 19 is formed.

Particles suspended in the air introduced through inlet 10 by the driving of fan 50A are negatively charged near discharge electrode 1. The negatively charged particles move toward collecting jig 12 because of electrostatic force, and are attracted and held by conductive coating, whereby the particles are collected on collecting jig 12. Here, since needle-shaped electrode is used as discharge electrode 1, it is possible to have charged particles attracted and held in a very narrow area corresponding to irradiation region 15 (as will be described later) irradiated by the light emitting element of collecting jig 12 opposite to discharge electrode 1. Consequently, in the detecting step as will be described later, it is possible to efficiently detect the attracted microorganisms.

The detecting mechanism included in detection chamber 5B includes: light emitting element 6 as a light source; light receiving element 9; and a collecting lens (or lenses) 13, provided in the direction of light reception by light receiving element 9, for collecting fluorescence generated by irradiation of airborne particles collected on collecting jig 12 by the collecting mechanism with light from light emitting element 6 to light receiving element 9. It may further include: a lens (or lenses) provided in a direction of light emission by light emitting element 6, for collimating the light beams from light emitting element 6 or to adjust the light beams to a prescribed width; an aperture; and a filter (or filters) for preventing entrance of irradiating light beam to light receiving element 9. Conventional configurations may be applied to these components. Collecting lens 13 may be formed of plastic resin or glass.

Preferably, at least the inner side of detection chamber 5B is painted black or treated with black alumite. This prevents reflection of light from the inner wall surface as a cause of stray light. Though the material of collection chamber 5A and detection chamber 5B is not specifically limited, preferably, plastic resin, aluminum, stainless steel or a combination of these may be used. Inlet 10 and outlet 11 of case 5 have circular shape with the diameter of 1 mm to 50 mm. The shape of inlet 10 and outlet 11 is not limited to a circle, and it may be an ellipse or a rectangle.

Light emitting element 6 is similar to that of detection apparatus 100A. Light beams emitted from light emitting element 6 are collected on a surface of collecting jig 12, and form irradiation region 15 on collecting jig 12. The shape of irradiation region 15 is not specifically limited, and it may have a circular, elliptical or rectangular shape. Though the size of irradiation region 15 is not specifically limited, preferably, the diameter of a circle, the longer side length of an ellipse or the length of one side of a rectangle is in the range of about 0.05 mm to 50 mm.

Light receiving element 9 is connected to signal processing unit 30 and outputs a current signal in proportion to the intensity of received light to signal processing unit 30. Therefore, fluorescence emitted from the particles that have been suspended in the introduced air, collected to the surface of collecting jig and irradiated with light from light emitting element 6, is received by light receiving element 9 and the intensity of received light is detected by signal processing unit 30.

A brush 60 for refreshing the surface of collecting jig 12 is provided at a position to touch the surface of collecting jig 12 in detection chamber 5B. Brush 60 is connected to a moving mechanism, not shown, controlled by measuring unit 40 and reciprocates on collecting jig 12 as represented by a double-sided arrow B in the figure. Consequently, dust and microorganisms deposited on collecting jig 12 are removed.

The heating mechanism is the same as that of detection apparatus 100A. In detection apparatus 100B, preferably, heater 91 is arranged on that surface of collecting jig 12 which is away from discharge electrode 1, as shown in FIG. 19. More preferably, heater 91 is surrounded by heat-insulating material as shown in FIG. 2B. Suitable heat insulating material includes glass epoxy resin.

A unit including collecting jig 12 and heater 91 will be referred to as a collection unit 12A here. Collection unit 12A is connected to a moving mechanism, not shown, controlled by measuring unit 40, and moves as indicated by double-sided arrow A in the figure, that is, from collection chamber 5A to detection chamber 5B and from detection chamber 5B to collection chamber 5A, through hole 5C′ formed in wall 5C. As already described, heater 91 may be arranged at a position allowing heating of airborne particles collected on collecting jig 12 and separated, at least at the time of heating, from the sensor equipment including light emitting element 6 and light receiving element 9 and, therefore, the heater may not be included in collection unit 12A and it may be provided at a different position. When the heating operation takes place in collection chamber 5A as will be described later, heater 91 may not be included in collection unit 12A but it may be fixed at a position, where collection unit 12A is set in collection chamber 5A, on a side of collecting jig 12 opposite to the sensor equipment including light emitting element 6 and light receiving element 9. By such an arrangement, at the time of heating, heater 91 is separated by collecting jig 12 from the sensor equipment including light emitting element 6 and light receiving element 9, whereby influence of heat on light emitting element 6, light receiving element 9 and the like can be prevented. Here, collection unit 12A may include at least collecting jig 12.

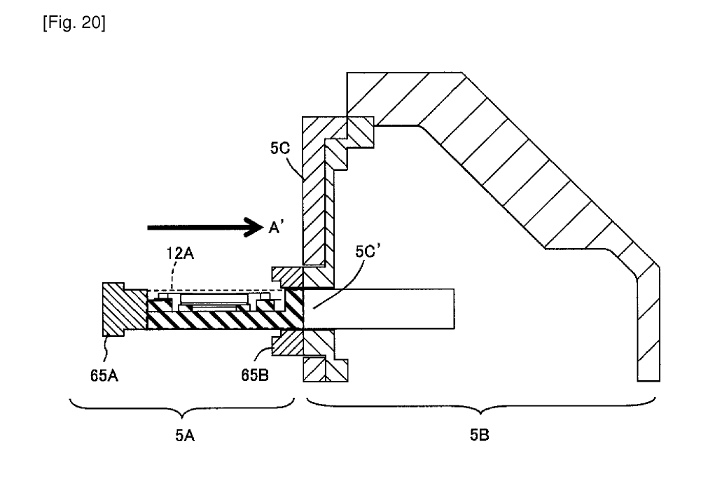

As shown in FIG. 20, at an end portion farthest from wall 5C of collection unit 12A, a cover 65A having upward and downward projections is provided. On a surface of wall 5C facing collection chamber 5A, around hole 5C′, an adapter 65B corresponding to cover 65A is provided. Adapter 65B has a recess that fits the projections of cover 65A. Therefore, cover 65A and adapter 65B are perfectly joined and cover hole 5C′. Specifically, when collection unit 12A moves in the direction of an arrow A′ of FIG. 20 from collection chamber 5A to detection chamber 5B through hole 5C′ and collection unit 12A comes to be fully received in detection chamber 5B, cover 65A is fit in adapter 65B, hole 5C′ is thus fully covered and detection chamber 5B is light-blocked. Thus, while the detecting operation is done in detection chamber 5B, entrance of light to detection chamber 5B is blocked.

The functional configuration of detection apparatus 100B for detecting airborne microorganisms utilizing the principle described with reference to FIGS. 5 to 14 is substantially the same as the functional configuration of detection apparatus 100A shown in FIG. 15. In the functional configuration of detection apparatus 100B, driving unit 48 drives, in place of heater 91, air introducing mechanism 50 and shutters 16A and 16B of detection apparatus 100A, fan 50A, heater 91, the mechanism, not shown, for reciprocating collection unit 12A and the mechanism, not shown, for reciprocating brush 60.

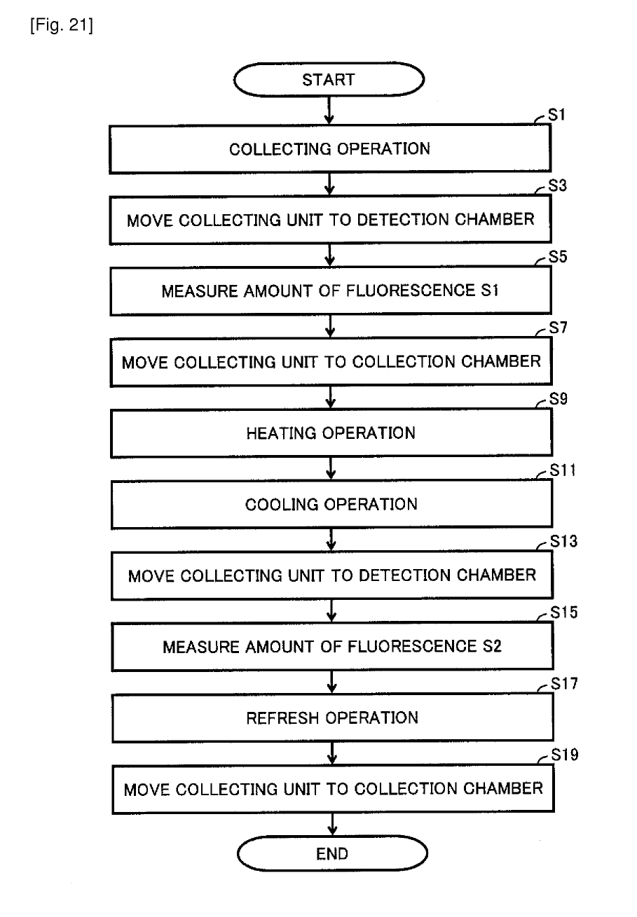

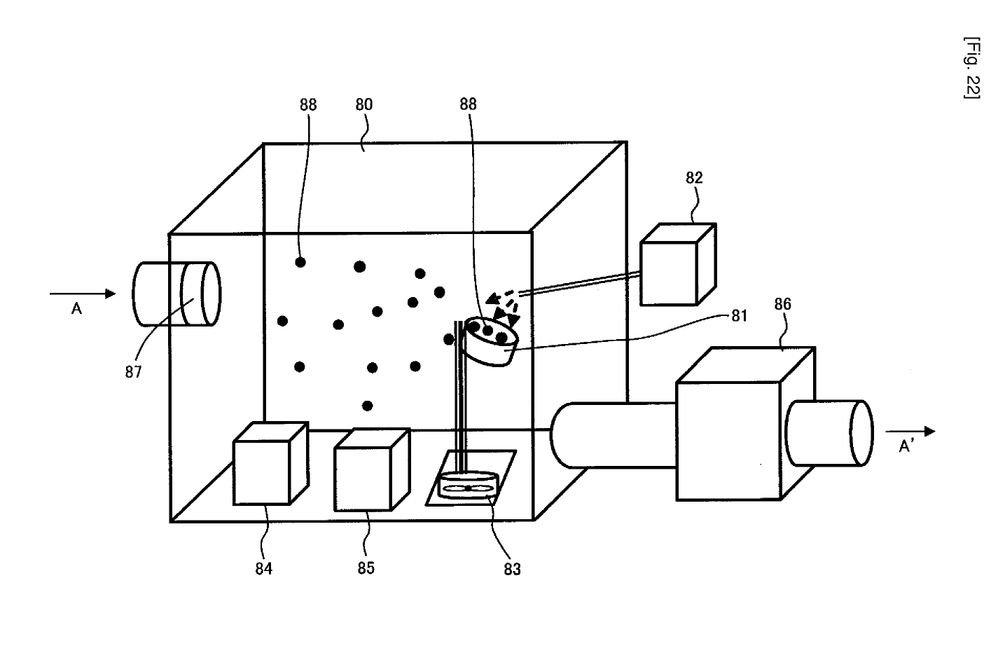

Specific operations in control unit 41 for calculating the amount of biological particles suspended in the air introduced to collection chamber 5A will be described with reference to the flowchart of FIG. 21. Here, as the amount of particles of biological origin, it is assumed that concentration of microorganisms suspended in the air introduced to case 5 is calculated.

Referring to FIG. 21, when detection apparatus 100B is powered ON, at step S1, a collecting operation is done in collection chamber 5A, for the time period DeltaT1 as a pre-defined collection time. Specific operations at step S1 are as follows. Control unit 41 outputs a control signal to driving unit 48 so that fan 50A is driven to feed air to collection chamber 5A. Particles in the air introduced to collection chamber 5A are negatively charged by discharge electrode 1, and because of the air flow caused by fan 50A and the electric field formed between discharge electrode 1 and coating 3 on the surface of collecting jig 12, the particles are collected to a narrow area corresponding to irradiation region 15 on the surface of collecting jig 12. When collection time DeltaT1 passes, control unit 41 ends the collecting operation, that is, ends the driving of fan 50A.

Thus, for the time period DeltaT1, external air is introduced to collection chamber 5A through inlet 10, and the particles in the air are collected for the time period DeltaT1 on the surface of collecting jig 12.

Next, at step S3, control unit 41 outputs a control signal to driving unit 48 to operate the mechanism for moving collection unit 12A, and collection unit 12A is moved from collection chamber 5A to detection chamber 5B. When the movement ends, at step S5, the detecting operation is done. As in detection apparatus 100A, at step S5, control unit 41 causes light emitting element 6 to emit light, and causes light receiving element 9 to receive light, for a defined measurement time DeltaT2. The light from light emitting element 6 is directed to irradiation region 15 on the surface of collecting jig 12, and fluorescence is emitted from collected particles. A voltage value in accordance with the fluorescence intensity F1 is input to measuring unit 40 and stored in storage unit 42. In this manner, an amount of fluorescence S1 before heating is measured.

The measurement time DeltaT2 may be set in advance in control unit 41, or it may be input or changed by an operation of, for example, switch 110, by a signal from PC 300 connected to communication unit 150 through cable 400, or by a signal from a recording medium attached to communication unit 150.

At this time, a separate light emitting element such as an LED (not shown) may be provided, light emitted from this element and reflected from a reflection region (not shown), at which particles are not collected, on the surface of collecting jig 12 may be collected by a separate light receiving element (not shown), the intensity of received light may be used as a reference value I0 and the value F1/I0 may be stored in storage unit 42. By calculating the ratio to reference value I0, it becomes advantageously possible to compensate for the fluctuation of fluorescence intensity derived from environmental conditions such as moisture and temperature of light emitting element or light receiving element, or from variation in characteristics caused by deterioration or aging.

When the measuring operation at step S5 ends, at step S7, control unit 41 outputs a control signal to driving unit 48 so that the mechanism for moving collection unit 12A is moved, and collection unit 12A is moved from detection chamber 5B to collection chamber 5A. When the movement ends, at step S9, heating operation is done. At step S9, as in detection apparatus 100A, control unit 41 causes heater 91 to heat for the predefined heating time DeltaT3. The heating temperature at this time is defined beforehand.

After the heating operation, at step S11, a cooling operation takes place. At step S11, control unit 41 outputs a control signal to driving unit 48 to cause fan 50A to rotate in reverse direction for a prescribed cooling time. Collecting unit 12A is cooled as external air is taken. Heating time DeltaT3, the heating temperature and the cooling time may be set in advance in control unit 41, or may be input or changed by an operation of, for example, switch 110, by a signal from PC 300 connected to communication unit 150 through cable 400, or by a signal from a recording medium attached to communication unit 150.

After collection unit 12A is moved to collection chamber 5A at step S7, the heating operation and cooling operation are done in collection chamber 5A, and after cooling, collection unit 12A is moved to detection chamber 5B. Therefore, at the time of heating, heater 91 is positioned at a distance separated from the sensor equipment including light emitting element 6 and light receiving element 9 and also separated by wall 5C and, therefore, influence of heat of light emitting element 6 and light receiving element 9 can be prevented. Since heater 91 is in collection chamber 5A separated also by wall 5C and the like from the sensor equipment including light emitting element 6 and light receiving element 9 at the time of heating, heater 91 may not necessarily be positioned on the surface away from discharge electrode 1 of collection unit 12A, that is, the surface away from light emitting element 6 and light receiving element 9 when collection unit 12A moves to detection chamber 5B, but it may be on a surface close to discharge electrode 1.

When the heating operation at step S9 and the cooling operation at step S11 end, at step S13, control unit 41 outputs a control signal to driving unit 48 so that the mechanism for moving collection unit 12A is operated, and collection unit 12A is moved from collection chamber 5A to detection chamber 5B. After the movement ends, at step S15, the detecting operation is done again. The detecting operation at step S15 is the same as the detecting operation at step S5. A voltage value at step S15 in accordance with the fluorescence intensity F2 is input to measuring unit 40 and stored in storage unit 42. In this manner, an amount of fluorescence S2 after heating is measured.

After the amount of fluorescence S2 after heating is measured at step S15, a refreshing operation of collecting unit 12A is done at step S17. At step S17, control unit 41 outputs a control signal to driving unit 48 to move the mechanism for moving brush 60, so that brush 60 reciprocates on the surface of collection unit 12A for a prescribed number of times. After the end of refreshing operation, at step S19, control unit 41 outputs a control signal to driving unit 48 to move the mechanism for moving collection unit 12A, and collection unit 12A is moved from detection chamber 5B to collection chamber 5A. Thus, the next collecting operation (S1) can be started immediately if a start instruction is received.

Calculating unit 441 calculates the difference between stored fluorescent intensities F1 and F2 as the amount of increase DeltaF. As in detection apparatus 100A, the concentration of particles of biological origin, obtained using the calculated amount of increase DeltaF and the correspondence relation (FIG. 17) between the amount of increase DeltaF and the concentration of particles of biological origin (particle concentration) stored beforehand, is calculated as the concentration of particles of biological origin in the air introduced to collection chamber 5A in time period DeltaT1. The calculated concentration of biological particles or microorganisms among the collected particles is output from control unit 41 to display unit 45 and displayed in the similar manner as in detection apparatus 100A (FIGS. 18A, 18B).

As described above, in detection apparatus 100B, collection chamber 5A and detection chamber 5B are sectioned and collection unit 12A moves between the chambers for collection and detection. Therefore, it is possible to perform collection and detection continuously. Further, collecting jig 12 is heated in collection chamber 5A, cooled and thereafter moved to detection chamber 5B, as described above. Therefore, influence of heat on the sensors and the like in detection chamber 5B can be prevented.

Further, in detection apparatus 100B, when collection unit 12A moves from collection chamber 5A for the collecting step to detection chamber 5B for the detecting step, the cover provided on collection unit 12A closes hole 5C′ of wall 5C. Therefore, entrance of external light to detection chamber 5B is blocked. Thus, stray light caused, for example, by scattering on airborne particles during fluorescence measurement can be reduced, and accuracy of measurement can be improved.

Though collection chamber 5A and detection chamber 5B are provided as chambers partitioned by wall 5C in detection apparatus 100B, it is also possible to provide a collecting device and a detecting device as fully separated bodies, and to have collection unit 12A moved therebetween, or to have collection unit 12A set to each device. In that case, heating of collecting jig 12 may be performed at a position outside the detecting device, separate from the sensor equipment including light emitting element 6 and light receiving element 9. By way of example, heating may be performed in the heating device corresponding to collection chamber 5A as described above, or at a position not in the collecting device or in the detecting device (for example, during movement from the collecting device to the detecting device). Heater 91 may be included in collection unit 12A or may be provided at a position to perform heating outside of the detecting device. Further, the collecting device and the detecting device may be used not as a set but each as a single device corresponding to collection chamber 5A or a single device corresponding to detection chamber 5B. In that case, the device used is adapted to include functions corresponding to signal processing unit 30, measuring unit 40 and the like.

Further, in detection apparatus 100B, one collection unit 12A is provided, and by reciprocation indicated by the double-sided arrow A, the unit moves to and from collection chamber 5A and detection chamber 5B. As another example, two or more collection units 12A may be provided on a turntable and moved between collection chamber 5A and detection chamber 5B as the table turns. In such a configuration, it is possible to position one of the plurality of collection units in collection chamber 5A and positioning another in detection chamber 5B, thereby to perform the collecting operation and the detecting operation in parallel. Such a configuration enables continuous collecting operations and continuous detecting operations in parallel.

In the second embodiment, description is made assuming that the air purifier shown in FIG. 1 functions as detection apparatus 100B. It is noted, however, that detection apparatus 100B may be used by itself.

The present inventors used the above described detection apparatus to measure an amount of particles of biological origin suspended in the air to verify the above described matters, as will be described hereinafter.

### Example 1

(1) Measurement Instrument

The present inventors used a detection apparatus 85 similar in structure to the FIG. 19 detection apparatus 100B to examine a correlation between concentration of airborne Penicillium particles and a value as measured by detection apparatus 85. Detection apparatus 85 was provided with collection chamber 5A having a size of 125 mm×80 mm×95 mm, and fan 50A having an aspiration ability of 20 litters/min. Light emitting element 6 was embodied by a semiconductor laser emitting laser light having a wavelength of 405 nm, and light receiving element 9 was embodied as a pin photodiode. Specifically, the detection apparatus measured a voltage value of signal processing unit 30. The voltage value represents an amount of light received by light receiving element 9, as detected by signal processing unit 30 from a signal of a current proportional to an amount of light received input from light receiving element 9.

FIG. 22 schematically shows a configuration of the instrument used by the present inventors for measurement. With reference to FIG. 22, for measurement, the present inventors arranged in an acrylic box 80 having a volume of 1 m³ a culture medium 81 having Penicillium incubated therein, an outlet of an air blowing device 82, an air blowing fan 83, detection apparatus 85, and a particle counter 84. Box 80 has two holes, one provided with a HEPA filter 87, and the other provided with a pump 86.

(2) Procedure of Measurement

The present inventors used the above measurement instrument to perform measurement in the following procedure:

<STEP1> Pump 86 is operated to aspirate air in box 80 in a direction indicated in FIG. 22 by an arrow A′. This draws air outside box 80 in a direction indicated in FIG. 22 by an arrow A, and passes the air through HEPA filter 87 and thus introduces the air into box 80. Pump 86 is continuously operated for several minutes and thereafter it is confirmed with particle counter 84 that there does not exist any particle having a diameter of 0.5 micrometer or larger, and then pump 86 is stopped.

<STEP2> Air blowing device 82 is operated to blow air therefrom to a surface of culture medium 81. This allows Penicillium spores 88 formed on the surface of culture medium 81 to fly in the air. At the time, fan 83 is also operated. This disperses Penicillium spores 88 in box 80 substantially uniformly.

<STEP3> Particle counter 84 is used to measure an amount N1 of Penicillium spores in box 80 before detection (STEP4).

<STEP4> Detection apparatus 85 is operated in a procedure similar to that shown in the FIG. 21 flowchart to measure Penicillium spores. More specifically, Penicillium spores in box 80 are measured through the following operations:

(STEP4-1) Detection apparatus 85 has collecting jig 12 moved to collection chamber 5A;

(STEP4-2) Fan 50 is operated and a voltage of 10 kV is applied between collecting jig 12 and discharge electrode 1 to introduce Penicillium spores 88 in box 80 into collection chamber 5A and thus collect them on a surface of collecting jig 12;

(STEP4-3) After such collection for 15 minutes, fan 50 is stopped and collecting jig 12 is moved from collection chamber 5A to detection chamber 5B;

(STEP4-4) Collecting jig 12 has the surface exposed to blue light of 405 nm emitted from a semiconductor laser or light emitting element 6;

(STEP4-5) Penicillium spores collected on the surface of collecting jig 12 emit amount of fluorescence S1, which is received by light receiving element 9 and its voltage value is stored in a personal computer (not shown) connected to detection apparatus 85;

(STEP4-6) Collecting jig 12 is moved from detection chamber 5B to collection chamber 5A;

(STEP4-7) A microceramic heater or the like embodying heater 91 is operated to heat the surface of collecting jig 12 at 200° C. for five minutes;

(STEP4-8) Heater 91 is stopped from operating, and fan 50 is operated for cooling for three minutes;

(STEP4-9) collecting jig 12 is moved from collection chamber 5A to detection chamber 5B, and, similarly as done through STEP4-2 to STEP4-5, amount of fluorescence S2 received by light receiving element 9 is measured and its voltage value is stored in the personal computer; and

(STEP4-10) A difference DeltaF between the voltage values measured before and after the heating is calculated as a value detected by detection apparatus 85.

<STEPS> Particle counter 84 is used to measure an amount N2 of Penicillium spores in box 80 after detection (STEP4), and from amounts N1 and N2 (for example an average value is calculated and) the amount of Penicillium spores in box 80 at the time of the detection is obtained, and it is divided by the volume of box 80 (of 1 m³) to calculate the concentration N of Penicillium spores in box 80 at the time of the detection (unit: 10,000 spores/m³).

(3) Result of Measurement

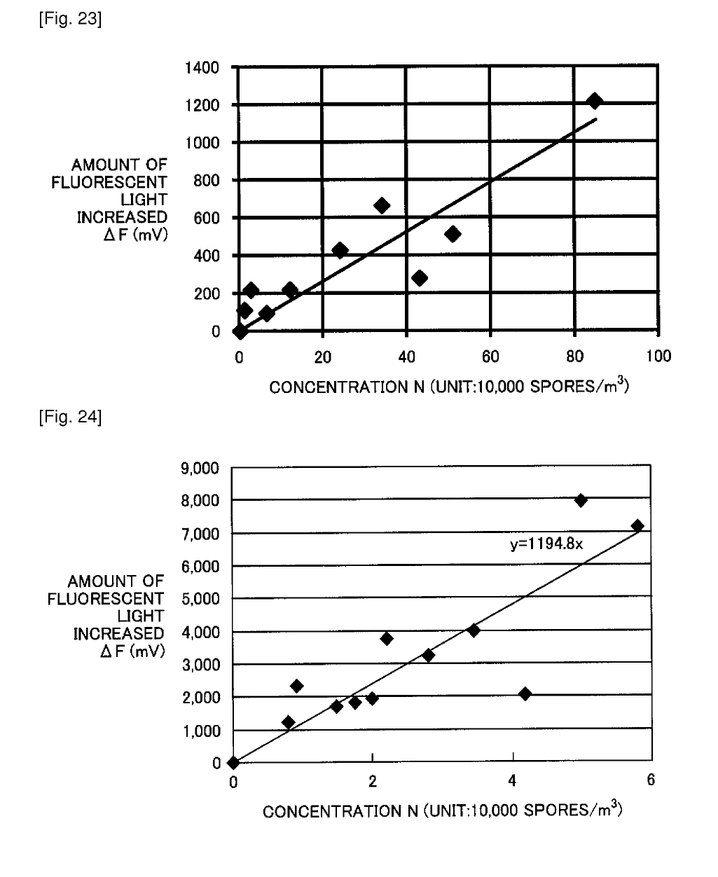

FIG. 23 shows a result of measurement in example 1. The present inventors obtained measurements in the above procedure for different concentrations N of Penicillium in box 80 such that, for each measurement, the surface of collecting jig 12 was refreshed with a glass fiber brush or collecting jig 12 used was replaced with a new collecting jig 12. A resultant measurement was plotted, as shown in FIG. 23 having an axis of abscissas representing a resultant measurement of concentration N of Penicillium in box 80 at the time of the detection and an axis of ordinates representing a value detected by detection apparatus 85, i.e., voltage difference DeltaF before and after the heating. The FIG. 23 measurement reveals that there is a linear correlation therebetween. It has thus been verified that the present detection apparatus described in the above embodiment allows microorganisms in the form of particles of biological origin to be detected with precision.

### Example 2

The present inventors employed a measurement instrument and procedure similar to those of example 1 to similarly obtain a measurement for cedar pollen. Note that in example 2, the measurement was performed such that culture medium 81 of example 1 having Penicillium incubated therein was replaced with a cylindrical pollen spray device which has one end provided with a filter and has opposite ends open.

In STEP2 described above, air blowing device 82 is operated to blow air externally of the cylinder from an end closer to the filter toward the interior of the cylinder to the pollen spray device with pollen introduced therein. This causes the pollen in the cylinder to fly in the air.

FIG. 24 shows a result of measurement in example 2. Similarly as done in FIG. 23, a resultant measurement is plotted, as shown in FIG. 24 having an axis of abscissas representing a resultant measurement of concentration N of cedar pollen in box 80 at the time of the detection and an axis of ordinates representing a value detected by detection apparatus 85, i.e., voltage difference DeltaF before and after the heating. The FIG. 24 measurement reveals that there is a linear correlation therebetween. It has thus been verified that the present detection apparatus described in the above embodiment allows pollen in the form of particles of biological origin to be detected with precision.

Furthermore, from examples 1 and 2, it has been verified that the present detection apparatus can detect with precision particles of biological origin, including microorganisms and pollen, that perform biotic activity, or a portion thereof, that are of sizes allowing the particles or a portion thereof to be airborne.

Although the present invention has been described and illustrated in detail, it is clearly understood that the same is by way of illustration and example only and is not to be taken by way of limitation, the scope of the present invention being interpreted by the terms of the appended claims.

### REFERENCE SIGNS LIST

1 discharge electrode
2 high-voltage power supply
3 coating
4 support board
5 case
5A collection chamber
5B detection chamber
5C wall
5C′ hole
6 light emitting element
7 lens
8 aperture
9 light receiving element
10 inlet
10A light shielding portion
10a, 10b light shielding plates
11 outlet
11A light shielding portion
12 collecting jig
13 collecting lens
14 filter
15 irradiation region
16A, 16B shutters
20 collection sensor mechanism
30 signal processing unit
34 current-voltage converting circuit
35 amplifying circuit
40 measuring unit
41 control unit
42 storage unit
43 clock generating unit
44 input unit
45 display unit
46 external connection unit
48 driving unit
50 air introducing mechanism
50A, 83 fan
51 recess
71-78 curves
80 box
81 culture medium
82 air blow device
84 particle counter
86 pump
87 HEPA filter
91 heater
85,100, 100A, 100B detection apparatuses
110 switch
130 display panel
150 communication unit
300 PC
400 cable
411 calculating unit

1.-15. (canceled)

16. A detection apparatus for detecting airborne particles of biological origin, comprising:
a light emitting element;
a light receiving element for receiving fluorescence; and
a calculating unit for calculating, based on an amount of fluorescence received by said light receiving element when air introduced to said detection apparatus is irradiated with light emitted from said light emitting element, an amount of particles of biological origin in said introduced air,
wherein said calculating unit calculates, based on a change in the amount of received light before and after heating said particles, said amount of particles in said introduced air.

17. The detection apparatus according to claim 16, further comprising a heater for heating said particles.

18. The detection apparatus according to claim 17, further comprising a control unit for controlling an amount of heating by said heater.

19. The detection apparatus according to claim 18, further comprising an input unit for inputting an instruction to said control unit.

20. The detection apparatus according to claim 16, wherein said calculating unit calculates, based on said change in the amount of received light, and on a relation between the amount of change in fluorescence and the amount of particles of biological origin stored in advance, said amount of particles of biological origin in said introduced air.

21. The detection apparatus according to claim 16, further comprising:
a collecting member; and
a collecting mechanism for collecting particles in said introduced air by said collecting member, wherein
said calculating unit calculates, based on the amount of received fluorescence from the collecting member irradiated with light emitted from said light emitting element, said amount of particles of biological origin collected by said collecting member.

22. The detection apparatus according to claim 21, wherein said light emitting element is arranged such that light is emitted in a direction toward said collecting member.

23. The detection apparatus according to claim 21, further comprising a heater for heating said collecting member, wherein said calculating unit calculates, based on a change in the amount of received light before and after heating of said collecting member, said amount of particles of biological origin collected by said collecting member.

24. The detection apparatus according to claim 21, further comprising:
a collection chamber housing said collecting mechanism;
a detection chamber separated from said collection chamber and housing said light emitting element and said light receiving element; and
a moving mechanism for moving said collecting member positioned in said collection chamber to said detection chamber, and for moving said collecting member positioned in said detection chamber to said collection chamber.

25. The detection apparatus according to claim 21, further comprising a cleaning unit for cleaning said collecting member.

26. The detection apparatus according to claim 16, further comprising a display unit for displaying a result of calculation by said calculating unit as a result of measurement.

27. The detection apparatus according to claim 16, wherein said light emitting element emits light in a wavelength range that can excite substance in a living organism.

28. The detection apparatus according to claim 27, wherein said light emitting element emits light in a wavelength range of 300 nm to 450 nm.

29. A method of detecting particles of biological origin collected by a collecting member, comprising the steps of:
measuring amount of fluorescence of said collecting member before heating, irradiated with light emitted from a light emitting element;
measuring amount of fluorescence of said collecting member after heating, irradiated with light emitted from said light emitting element; and
calculating an amount of particles of biological origin collected by said collecting member, based on an amount of change in said amount of fluorescence measured from said collecting member before heating and said amount of fluorescence measured from said collecting member after heating.
